(19) United States
(12) Patent Application Publication
(10) Pub. No.: US 2014/0277201 A1
(43) Pub. Date: Sep. 18, 2014

(54) Positioning Rod for a Hollow Pedicle of a Minimally Invasive Surgery

(71) Applicant: Chih-Hsuan Wei, Chiayi City (TW)

(72) Inventor: Chih-Hsuan Wei, Chiayi City (TW)

(21) Appl. No.: 13/845,246

(22) Filed: Mar. 18, 2013

(51) Int. Cl. A61B 17/88

(52) U.S. Cl. CPC A61B 17/88
USPC 606/86 A

(57) ABSTRACT

A positioning rod of a hollow pedicle for a minimally invasive surgery contains a hollow tube section and a circular clamping portion, and the clamping portion including a hole defined at a central portion thereof so as to fit the circular clamping portion on a top end of the hollow tube section. The circular clamping portion is made of plastic material. The color of the circular clamping portion is variable so as to provide a distinguishing function.

### BACKGROUND OF THE INVENTION

1. Field of the Invention

The present invention relates to a positioning rod of a hollow pedicle for a minimally invasive surgery, and more particularly to a positioning rod which is fixed on a spine so that surgery operator distinguishes a surgery position and erects a bone screw easily.

2. Description of the Prior Art

In a conventional spine surgery, a bone screw is erected after scraping cartilage and it is fixed on spines on two sides of a surgery position so that the spines repairs autologously and facilitates correctional rehabilitation.

However, in a scraping surgery or as erecting the bone screws, the surgery operator has to distinguish a surgery position by ways of eye identification, thus causing inaccuracy and inconvenience.

The present invention has arisen to mitigate and/or obviate the afore-described disadvantages.

### SUMMARY OF THE INVENTION

The primary object of the present invention is to provide a positioning rod of a hollow pedicle for a minimally invasive surgery which is capable of overcoming the shortcomings of the conventional positioning rod.

Another object of the present invention is to provide a positioning rod of a hollow pedicle for a minimally invasive surgery which is fixed on a spine so that a surgery operator distinguishes a surgery position and erects a bone screw easily.

To obtain the above objectives, a positioning rod of a hollow pedicle for a minimally invasive surgery contains a hollow tube section and a circular clamping portion, and the clamping portion including a hole defined at a central portion thereof so as to fit the circular clamping portion on a top end of the hollow tube section.

The circular clamping portion is made of plastic material.

The color of the circular clamping portion is variable so as to provide a distinguishing function

### BRIEF DESCRIPTION OF THE DRAWINGS

FIG. 1 is a perspective view of the assembly of a positioning rod of a hollow pedicle for a minimally invasive surgery according to a preferred embodiment of the present invention.

FIG. 2 is a cross sectional view of an application of the positioning rod of the hollow pedicle for the minimally invasive surgery according to the preferred embodiment of the present invention.

FIG. 3 is a cross sectional view of another application of the positioning rod of the hollow pedicle for the minimally invasive surgery according to the preferred embodiment of the present invention.

### DETAILED DESCRIPTION OF THE PREFERRED EMBODIMENTS

The present invention will be clearer from the following description when viewed together with the accompanying drawings, which show, for purpose of illustrations only, the preferred embodiment in accordance with the present invention.

With reference to FIG. 1, a positioning rod 1 of a hollow pedicle for a minimally invasive surgery according to a preferred embodiment of the present invention comprises: a hollow tube section 11 and a circular clamping portion 12, and the circular clamping portion 12 including a hole 121 defined at a central portion thereof so as to fit the circular clamping portion 12 on a top end of the hollow tube section 11.

The circular clamping portion 12 is made of plastic material.

A color of the circular clamping portion 12 is variable so as to provide a distinguishing function.

As shown in FIGS. 2 and 3, in operation, guiding posts 2 are inserted into spines 3 on two sides of a surgery position, and hollow tube sections 11 of the positioning rods 1 are inserted into the guiding posts 2 so that the positioning rods 1 are implanted into the spines 3, hence circular clamping portions 12 of the positioning rods 1 distinguish the surgery position quickly. When desiring to implant bone screws after a surgery, the positioning rods 1 are used to distinguish positions where implants the bone screws, and then the positioning rods 1 are removed so as to implant the bone screws, thus enhancing implanting accuracy and convenience.

Thereby, the positioning rods 1 are fixed on the spines 3 so that the surgery positions are distinguished clearly, and the bone screws are erected easily.

While we have shown and described various embodiments in accordance with the present invention, it is clear to those skilled in the art that further embodiments may be made without departing from the scope of the present invention.

What is claimed is:

1. A positioning rod of a hollow pedicle for a minimally invasive surgery comprising:
a hollow tube section and a circular clamping portion, and the clamping portion including a hole defined at a central portion thereof so as to fit the circular clamping portion on a top end of the hollow tube section.

2. The positioning rod of the hollow pedicle for the minimally invasive surgery as claimed in claim 1, wherein the circular clamping portion is made of plastic material.

3. The positioning rod of the hollow pedicle for the minimally invasive surgery as claimed in claim 1, wherein the color of the circular clamping portion is variable so as to provide a distinguishing function.
